SEQUENCE LISTING (8 sequences)

SEQ ID NO: 1 - DNA, 44 bp, synthetic construct
  source 1..44: mol_type = other DNA
tgctccagttcggtcagtttgcccgcattgcattagcattagga

SEQ ID NO: 2 - DNA, 19 bp, synthetic construct
  source 1..19: mol_type = other DNA
atgctccagttcggtcagt

SEQ ID NO: 3 - DNA, 50 bp, synthetic construct
  source 1..50: mol_type = other DNA
tttttatccgacgttggcatgtgagtatgttcactgaccgaactggagca

SEQ ID NO: 4 - DNA, 20 bp, synthetic construct
  source 1..20: mol_type = other DNA
ctcacatgccaacgtcggat

SEQ ID NO: 5 - DNA, 44 bp, synthetic construct; identical to SEQ ID NO: 1
  source 1..44: mol_type = other DNA

SEQ ID NO: 6 - DNA, 19 bp, synthetic construct
  source 1..19: mol_type = other DNA
atgctccagttcggtcagt

SEQ ID NO: 7 - DNA, 39 bp, synthetic construct
  source 1..39: mol_type = other DNA
ccaagacgcgccggtctgttcactgaccgaactggagca

SEQ ID NO: 8 - DNA, 32 bp, synthetic construct
  source 1..32: mol_type = other DNA
gtcagtgaacagaccggcgcgtcttggttttt

(19) United States
(12) Patent Application Publication
(10) Pub. No.: US 2025/0270625 A1
(43) Pub. Date: Aug. 28, 2025

(54) NUCLEIC ACID DETECTION IN A PCR USING A TARGET SEQUENCE-UNSPECIFIC MODULAR REPORTER COMPLEX AND ELECTROCHEMICAL DETECTION

(71) Applicants: ALBERT-LUDWIGS-UNIVERSITÄT FREIBURG, Freiburg (DE); HAHN-SCHICKARD-GESELLSCHAFT FÜR ANGEWANDTE FORSCHUNG E. V., Stuttgart (DE)

(72) Inventors: Felix VON STETTEN, Stuttgart (DE); Tamara PFAFF, Freiburg (DE); Michael LEHNERT, Stuttgart (DE); Martin TROTTER, Stuttgart (DE); Nadine BORST, Stuttgart (DE); Lisa BECHERER, Freiburg (DE); Helena GMOSER

(21) Appl. No.: 18/858,489

(22) PCT Filed: Apr. 21, 2023
(86) PCT No.: PCT/EP2023/060527

§ 371 Date: Apr. 3, 2025

(30) Foreign Application Priority Data
Apr. 22, 2022 (EP) 22169463.1

(51) Int. Cl. C12Q 1/6818; C12Q 1/6834; C12Q 1/686

(52) U.S. Cl. CPC C12Q 1/6818; C12Q 1/6834; C12Q 1/686

(57) ABSTRACT

The invention relates to a method for detecting at least one target nucleic acid sequence by means of a method for detecting at least one target nucleic acid sequence, comprising the steps of: i. Providing at least one target sequence-unspecific modular reporter complex comprising at least one label and at least two oligonucleotides, namely, i. a base strand comprising, 1. at least one mediator binding site, 2. at least one signal initiation oligo binding site, ii. at least one signal initiation oligo, wherein optionally the signal initiation oligo binding site of the base strand and the at least one signal initiation oligo are hybridized to each other but not covalently bonded and together form a signal complex, j. Providing at least one mediator probe, wherein the mediator probe comprises an oligonucleotide having at least one probe sequence and at least one mediator sequence, wherein the at least one probe sequence exhibits an affinity for at least one target nucleic acid sequence, and the at least one mediator sequence exhibits an affinity for at least one mediator binding site on the base strand of the at least one target sequence-unspecific modular reporter complex, k. PCR amplification of at least one nucleic acid sequence, I. Binding of a probe sequence of at least one mediator probe to at least one target nucleic acid sequence, m. Cleavage of the probe sequence of the at least one mediator probe bound to the at least one target nucleic acid sequence by a PCR polymerase, wherein the mediator sequence is released, n. Binding of at least one released mediator sequence to a mediator binding site of the at least one target sequence-unspecific modular reporter complex, o. Extension of the sequence of at least one mediator sequence bound to a mediator binding site by a PCR polymerase, wherein the bond is broken or prevented by hybridization of the at least one signal initiation oligo binding site and the at least one signal initiation oligo, thereby initiating a signal change, p. Detection of at least one signal change as evidence of the at least one target nucleic acid sequence.

### TECHNICAL BACKGROUND

In quantitative detection reactions of PCR products such as real-time PCR (qPCR) or digital PCR (dPCR), target sequences are mainly detected either by dyes that intercalate into the DNA but bind unspecifically to all DNA double strands present, or by DNA probes that only bind a specific DNA target sequence. These DNA probes generate an optical signal change directly (e.g. Taqman probes) or indirectly (mediator probes in combination with universal reporter molecules) as a result of their cleavage. Optical detectors thereby detect the light emissions generated during the reaction outside the reaction vessel. These detection systems usually rely on light-absorbing and emitting fluorescent molecules. After excitation by light energy of a certain wavelength, these molecules emit energy in the form of higher wavelengths, which can be detected by detectors. A distinction is made between molecules whose light energy is to be detected in a specific wavelength range (fluorescence donor or fluorophore) and molecules that lead to a decrease in the fluorescence intensity of a fluorophore in close proximity (fluorescence acceptor or quencher). If the spatial proximity between fluorophore and quencher changes, the fluorescence signal changes accordingly, wherein a smaller distance always results in a higher degree of quenching (FRET quenching or contact quenching). The use of target sequence-specific DNA probes in combination with different fluorescent labels also enables the sensitive detection of a plurality of DNA sequences in one reaction (multiplex detection). This usually involves the use of a plurality of DNA probes or detection molecules that carry different fluorophore-quencher combinations, each of which emits a fluorescence signal in a specific wavelength range in the presence of a specific DNA sequence. Further developments of this multiplexing detection also enable the detection of a plurality of target sequences within a wavelength range by quantitatively observing the fluorescence values or combining them with other parameters (e.g. the readout temperature).

Two well-known one-piece target sequence-specific DNA probes for optical detection are Taqman probes and molecular beacons (Tan et al. 2004; Li et al. 2008; Holland et al. 1991; Rodríguez et al. 2005) These bind in a target sequence-specific manner and are cleaved during PCR, generating a signal. The disadvantage of these probes is their dependence on the target gene sequence, which means that only specific regions of a DNA target sequence, which must meet certain requirements, can be used. For example, attention must be paid to probe length, melting temperature, binding enthalpy, GC content, guanine quenching and complementary sequence fragments. In addition, their synthesis can be very demanding.

There are individual examples of (specific) electrochemical detection of target sequences in the liquid phase. More frequently, the detection of target sequences by hybridization reactions on functionalized electrodes is described.

Examples of detection in the liquid phase work according to the molecular beacon method. Molecular beacons comprise an oligonucleotide (oligo) with five to seven complementary bases at both ends and a terminal fluorophore or quencher (Tyagi and Kramer 1996). Instead of a fluorophore in a molecular beacon, an electroactive label is used and instead of the quencher, a molecule is used that complexes the electroactive molecule and thus prevents effective electron transfer. The complexing molecule can be dispensed with if the secondary structure of the molecular beacon forms a reversible binding region (in the form of an aptamer) for the electroactive molecule. In this case, the hybridization of the target sequence with the molecular beacon causes the release of the electroactive molecule.

The specific electrochemical detection of target DNA is often carried out using solid phase via specifically functionalized electrodes to which the amplification product or a probe hybridizes. Prior to the hybridization reaction, single-stranded DNA molecules are usually generated from double-stranded amplification products, for example by enzymatic digestion. For signal generation, either intercalating electroactive molecules or another hybridization probe modified with an electroactive molecule are then added.

DNA detection reactions for which electrode surfaces have been functionalized with universally usable DNA probes can be found in the literature. However, the target DNA is usually not amplified, so the sensitivity of the methods is low. Some methods require the target sequence to be present as a single strand, so either an initial single-strand generation step is required or the application is restricted to single-stranded RNA. The reaction times are also often significantly longer than would be expected for a DNA amplification reaction. According to our current knowledge, only one form of hybridization chain reaction is comparable to a PCR, for example, for which detection limits of 50 aM are achieved within 120 min according to the publication (Liu et al. 2017). This also requires a single-stranded target sequence that unfolds a first DNA probe (folded in a hairpin structure). Unfolded, the first probe can unfold a second universal probe (initially also present as a hairpin structure). This second probe is modified with an electroactive molecule. In the unfolded state, this second probe can hybridize to another surface-bound probe such that electroactive molecules are enriched on the electrode surface.

Patent WO2013079307A1 Bifunctional oligonucleotide probe for universal real-time multianalyte detection claims the Mediator probe technology system. In this system, a mediator probe is activated by extending a primer (auxiliary molecule 1) by means of a polymerase (auxiliary molecule 2) at the target sequence (target molecule). Due to the exonuclease activity of the polymerase, the mediator probe is cleaved and can then bind to the UR (mediator hybridization sequence). Here it acts as a primer after cleavage. As a result, it is extended by the polymerase and thus separates the fluorophore and quencher on the universal reporter, which leads to signal generation.

An example of two-part reporter systems can be found in patent WO2018114674A1 for the loop-mediated isothermal amplification method with mediator-displacement probes (MD LAMP), in which a universal reporter with at least one oligonucleotide and at least one fluorophore and quencher for a LAMP reaction is claimed. Until now, it was assumed that this only works because a LAMP, unlike a PCR, has a continuously uniform temperature, which quickly establishes and maintains an equilibrium, allowing individual molecules to bind to each other permanently and thus not generate a signal in the initial state.

Even with regard to detection on a solid phase, only one-part universal reporters that are attached to electrode surfaces have been described to date. The signal change has so far been caused by hybridization of one displaced mediator.

In multiplex variations, different wavelengths are generally used in optical detection reactions in order to distinguish individual target sequences in certain light ranges, the so-called detection channels, in a reaction. This restricts multiplexing, as only one target sequence can be detected per channel. The degree of multiplexing in most commercial devices is therefore limited to five or six channels.

An alternative approach to multiplexing is represented by patents with detection systems that are also based on a separation of signal generation and detection, such as the patent specification US20200087718A1 from Seegene for signal molecule-based detection using melting curve analysis. This patent claims the possibility of increasing the degree of multiplexing by using different lengths of the target sequence-unspecific signal molecule to generate different melting temperatures. A further divisional application (EP2708608) uses only the signal readout at predefined temperatures in contrast to the original patent, since the corresponding one-part signal molecules of different sequence lengths also show different fluorescence behavior at defined readout temperatures.

Another patent is the patent of the company Biorad (U.S. Pat. No. 9,921,154B2), which has only been granted in the USA. This also claims the detection of a plurality of target sequences in identical detection channels in a digital PCR, but according to current understanding can only be used if a significant multiple occupancy with DNA target sequences occurs per reaction space of a digital PCR, which thus lead to distinguishable signal clusters in the data space. The method according to the invention described herein has the advantage over this patent that it can also be used when there is no multiple occupancy.

An example of universal electrochemical detection can be found in patent specification DE102011056606B3 from FRIZ Biochem GmbH, which describes a universal detection method for electrochemical detection. A so-called signal oligo is used, which has a hairpin structure. The loop region contains a target DNA-specific sequence. The stem of the hairpin contains a universal sequence that can hybridize to the complementary universal solid phase probe and is modified for detection with an electroactive molecule. In the folded state, hybridization is not effective. When the signal initiation oligo hybridizes to the target sequence, it unfolds and the sequences of the stem form flaps. During a DNA amplification reaction, the exonuclease domain of the polymerase cuts the flap sequence such that it can hybridize more effectively with the solid phase probe.

A disadvantage of this method is that the signal oligo is modified with a relatively expensive electroactive molecule. Since the signal oligo also contains a target sequence-specific region, assay optimization can be expensive. The design of the signal oligo and solid phase probe is not trivial. By optimizing the melting temperatures, the solid phase probe must be prevented from already interacting with the still folded signal oligo. At the same time, the complementary parts of the strain must be prevented from hybridizing with each other again even after cleavage by the exonuclease activity. The patent specification therefore describes that the solid-phase probe should contain individual bases that are not complementary to the stem of the signal oligonucleotide in order to optimize the melting temperatures.

### Objective of the Invention

PCR is the gold standard method for amplifying individual DNA sequences and making them detectable. Either intercalating dyes or DNA probes are used to detect and quantify PCR products (DNA or cDNA in the case of RNA). However, intercalating dyes bind unspecifically to all double-stranded DNA molecules present, making direct sequence-specific detection in PCR impossible. DNA probes are signal-generating DNA sequences that are complementary to the respective sequence section of a PCR product and can therefore specifically detect and quantify it. This method is used in particular in real-time PCR and digital PCR. Currently, DNA probes themselves either have a biochemical modification to generate a signal in the presence of a DNA target sequence during PCR (e.g. Taqman probes) or activate a second detection molecule that generates a signal independently of the target sequence (e.g. mediator probes in combination with target sequence-unspecific universal reporters).

For detection, the method according to the invention preferably uses electrochemical methods for detecting DNA sequences in a PCR. These differ greatly in their methodology from the optical methods described so far, since a signal change is generated via a change in the electrical charge transfer at an electrode. This means that the signal change must be detected at the electrode solid phase. This has many advantages. For example, the signal detection is very sensitive and direct, which makes corresponding expensive optical detection systems superfluous. This is an advantage as the corresponding electrochemical detectors are significantly cheaper, more robust and easy to miniaturize. Until now, the lack of direct transferability between optical and electrochemical detection has posed a problem, as it is not sufficient to simply add a biosensor (e.g. a DNA sequence) to an electrode. Instead, all detection components must be adapted and other detection mechanisms must be used. This hurdle prevents the rapid transfer of detection systems between optical and electrochemical detection.

This highlights the urgent need for a new type of detection process and signaling molecule that overcomes these problems. In addition, this detection process should be based on a common basic process so that similar DNA sequence layouts can be used for optical or electrochemical detection to ensure rapid transfer between different detection methods.

The core of such a process would be a new type of modular detection molecule complex, which is target sequence-unspecific, flexible in design and easy to optimize and has the same performance characteristics as the current prior art describes for one-piece detection molecules. To this end, it should have similar sequence patterns in order to be able to transfer detection processes more easily between optical and electrochemical detection. So far, such a modular composition has failed in particular because it does not have covalent chemical bonds, which makes it unsuitable for PCR applications according to current assumptions of the prior art.

Once optimized, the reaction of the signal-activating or signal-initiating oligonucleotides, in particular the hybridization reaction on the solid-phase surface, should also be easily and reproducibly transferable to other target sequences. The universal character of the surface modification should make it possible to leave the sequences involved in the surface reactions unchanged, even if these surfaces are to be used for the detection of other target sequences. The target sequence-specific reactions should therefore all take place in the liquid phase, so that only the reagents and oligonucleotides of the liquid phase need to be adapted, newly developed and optimized for the development of a new nucleic acid detection system. The surfaces and their modification (e.g. the immobilization of oligonucleotides on electrode surfaces), on the other hand, should be able to be produced in large batches and thus at low cost. These measures are intended to save development effort (time and costs).

### SUMMARY OF THE INVENTION

The objective according to the invention is solved by the features of the independent claims. Advantageous embodiments of the invention are described in the dependent claims.

Thus, in one aspect, the invention relates to a method for detecting at least one target nucleic acid sequence, comprising the steps of:
a. Providing at least one target sequence-unspecific modular reporter complex comprising at least one label and at least two oligonucleotides, namely
   i. a basic strand, comprising
      1. at least one mediator binding site
      2. at least one signal initiation oligo binding site
   ii. at least one signal initiation oligo
wherein optionally the signal initiation oligo binding site of the base strand and the at least one signal initiation oligo are hybridized with each other but not covalently bonded and together form a signal complex.

b. Providing at least one mediator probe, wherein the mediator probe comprises an oligonucleotide having at least one probe sequence and at least one mediator sequence, wherein
the at least one probe sequence exhibits an affinity for at least one target nucleic acid sequence, and the at least one mediator sequence exhibits an affinity for at least one mediator binding site on the base strand of the at least one target sequence-unspecific modular reporter complex,
c. PCR amplification of at least one nucleic acid sequence,
d. Binding a probe sequence of at least one mediator probe to at least one target nucleic acid sequence,
e. Cleavage of the probe sequence of the at least one mediator probe bound to the at sequence is released,
f. Binding of at least one released mediator sequence to a mediator binding site of the at least one target sequence-unspecific modular reporter complex,
g. Extension of the sequence of at least one mediator sequence bound to a mediator binding site by a PCR polymerase, wherein the bond of the hybridized at least one signal initiation oligo binding site and the at least one signal initiation oligo is broken, thereby initiating a signal change,
h. Detection of at least one signal change as evidence of the at least one target nucleic acid sequence.

In a further preferred embodiment, the method of detecting at least one target nucleic acid sequence comprises the following steps:
a. Providing at least one target sequence-unspecific modular reporter complex comprising at least one label, and
   at least two oligonucleotides, namely
      i. a basic strand comprising
         1. at least one mediator binding site
         2. at least one signal initiation oligo binding site
      ii. at least one signal initiation oligo
wherein optionally the signal initiation oligo binding site of the base strand and the at least one signal initiation oligo are hybridized with each other but not covalently bonded and together form a signal complex,
b. Providing at least one mediator probe, wherein the mediator probe comprises an oligonucleotide having at least one probe sequence and at least one mediator sequence, wherein
the at least one probe sequence exhibits an affinity for at least one target nucleic acid sequence, and the at least one mediator sequence exhibits an affinity for at least one mediator binding site on the base strand of the at least one target sequence-unspecific modular reporter complex,
c. PCR amplification of at least one nucleic acid sequence,
d. Binding a probe sequence of at least one mediator probe to at least one target nucleic acid sequence,
e. Cleavage of the probe sequence of the at least one mediator probe bound to the at sequence is released,
f. Binding of at least one released mediator sequence to a mediator binding site of the at least one target sequence-unspecific modular reporter complex,
g. Extension of the sequence of at least one mediator sequence bound to a mediator binding site by a PCR polymerase, wherein the bond of the at least one signal initiation oligo binding site and the at least one signal initiation oligo is broken and/or prevented, preferably via hybridization, thereby initiating a signal change,
h. Detection of at least one signal change as evidence of the at least one target nucleic acid sequence.

In a further preferred embodiment, the method of detecting at least one target nucleic acid sequence comprises the following steps:
a. Providing at least one target sequence-unspecific modular reporter complex comprising at least one label, and
   at least two oligonucleotides, namely
   i. a basic strand comprising
      1. at least one mediator binding site
      2. at least one signal initiation oligo binding site
   ii. at least one signal initiation oligo
wherein optionally the signal initiation oligo binding site of the base strand and the at least one signal initiation oligo are hybridized with each other but not covalently bonded and together form a signal complex,
b. Providing at least one mediator probe, wherein the mediator probe comprises an oligonucleotide having at least one probe sequence and at least one mediator sequence, wherein
the at least one probe sequence exhibits an affinity for at least one target nucleic acid sequence, and the at least one mediator sequence exhibits an affinity for at least one mediator binding site on the base strand of the at least one target sequence-unspecific modular reporter complex,
c. PCR amplification of at least one nucleic acid sequence,
d. Binding a probe sequence of at least one mediator probe to at least one target nucleic acid sequence,
e. Cleavage of the probe sequence of the at least one mediator probe bound to the at sequence is released,
f. Binding of at least one released mediator sequence to a mediator binding site of the at least one target sequence-unspecific modular reporter complex,
g. Extension of the sequence of at least one mediator sequence bound to a mediator binding site by a PCR polymerase, wherein the bond or hybridization of the at least one signal initiation oligo binding site and the at least one signal initiation oligo is either i) broken and/or prevented or ii) initiated and/or produced, thereby initiating a signal change,
h. Detection of at least one signal change as evidence of the at least one target nucleic acid sequence.

In embodiments of the method according to the invention (e.g. similar to that shown in FIG. 6 ii)), the signal oligo and the base strand are not hybridized with each other from the outset and do not yet form a signal complex in step a. The extension of the mediator on the base strand can take place in some such embodiments, e.g. in a separate chamber/cavity/well, such that the hybridization takes place, for example, in a (later) endpoint detection, only at a later time/in a later step, quasi retrospectively. In some of these embodiments, the signal change in step g. is therefore not a “signal change” but rather an altered signal compared to a control reaction with an intact mediator probe.

In embodiments of the method according to the invention, the signal change in step g. is therefore an altered signal, preferably an altered signal compared to a control reaction with an uncleaved mediator probe, which is initiated or present.

In embodiments, step g. comprises extending the sequence of at least one mediator sequence bound to a mediator binding site by a PCR polymerase, wherein the bond (and/or hybridization) of the at least one signal initiation oligo binding site and the at least one signal initiation oligo is either i) broken and/or prevented, or ii) initiated or enabled/established, whereby/such that an altered signal is elicited as compared to a control reaction with uncleaved mediator probe.

In embodiments, step g. comprises extending the sequence of at least one mediator sequence bound to a mediator binding site by a PCR polymerase, wherein the bond (hybridization) of the at least one signal initiation oligo binding site and the at least one signal initiation oligo is either i) broken and/or prevented or ii) initiated or enabled/established, thereby initiating a signal change. In embodiments, a signal change is an altered signal, preferably an altered signal compared to a control reaction with an uncleaved mediator probe.

Example embodiments are shown in FIGS. 6i-6v. In some embodiments, a signal change is generated by hybridizing a signal initiation oligo with an oligonucleotide, wherein preferably one of the two molecules comprises at least one label, in other embodiments, a signal change is generated by preventing or cleaving/breaking a hybridization. In certain embodiments, a signal change is achieved by changing the distance from at least one electroactive molecule to a surface as a result of the severing of the bond between the signal initiation oligo and the base strand. Here, an extension of the mediator causes the distance to the surface either to increase (FIG. 6iv) or decrease (FIG. 6v), which in both cases leads to a signal change.

In embodiments, step g. comprises extending the sequence of at least one mediator sequence bound to a mediator binding site by a PCR polymerase, wherein the bond of the at least one signal initiation oligo binding site and the at least one signal initiation oligo, optionally hybridized with each other, is broken and/or prevented, thereby initiating a signal change and/or an altered signal (e.g. compared to a control reaction).

In embodiments, in step a), the signal initiation oligo binding site of the base strand and the at least one signal initiation oligo are hybridized with each other, but not covalently bonded, and together form a signal complex.

In embodiments of the method according to the invention, a signal change is initiated in step g. by the separated at least one signal initiation oligo comprising at least one label and binding to at least one auxiliary oligo (preferably comprising a signal oligonucleotide binding site) which is immobilized on a solid phase. Non-limiting examples of the embodiments are shown in FIG. 6 iii).

In embodiments of the method according to the invention, in step g., a signal change is initiated by immobilizing one of the two oligonucleotides or both oligonucleotides of the at least one target sequence-unspecific modular reporter complex, namely
i. the base strand, and/or
ii. the at least one signal initiation oligo,
   to a fixed phase via one linker each.

The modular reporter system for target sequence-unspecific detection is a new invention that distinguishes itself from previously known detection systems through the flexible use of different oligonucleotides with different labels. Above all, the modular structure offers various advantages such as the possibility to adapt the design flexibly to specific device conditions, the uniformity of the signal generation reaction between different detection methods or the inexpensive, simple production as well as completely novel multiplex detection methods. The system and its functionality have not yet been described in a patent or in the literature on nucleic acid detection in a PCR.

The core of the invention is the modular target sequence-independent modular reporter complex (e.g. in FIG. 1) comprising non-covalently bonded oligonucleotides. This system has all the advantages of the various target sequence-specific detection systems as well as target sequence-unspecific detection molecules and combines them with an increased flexibility in the design of such detection molecules, which leads to completely new detection methods. Surprisingly, the system can be used in a PCR with cyclic temperature changes, and the associated melting of the DNA strands in each cycle, without any problems. Investigations by the inventors have shown that the modular system according to the invention even outperforms the systems of the prior art in terms of functionality. The significantly cheaper production can be achieved with at least as good performance parameters. A further advantage of the system according to the invention is the possibility of flexible adaptation of the universal probe according to the invention.

The modular system of the target sequence-independent modular reporter complex according to the invention comprises a basic strand. This has at least one binding site for a mediator (receptor complex) and at least one binding site for a signal initiation oligonucleotide (signal complex). The base strand and at least one signal initiation oligonucleotide together form a complex (e.g. FIG. 1). At least one of these strands has a label that can detect a structural change in this complex. The modular structure results from a plurality of possible signal initiation oligonucleotides (or “signal initiation oligos”) which can be combined with a base strand in different ways in different embodiments (see e.g. FIG. 5-7). The advantages of target gene-dependent systems of the prior art can be taken up by various possibilities for attaching signal initiation oligonucleotides and base strands to a solid phase (e.g. an electrode). Due to the modular design, a much higher number of labels can be attached per complex than is possible with current one-piece detection molecules.

This novelty of the modular detection complex opens up new possibilities for detection reactions based on a basic reaction which is described below as an example.

During a PCR detection reaction, a mediator probe binds to the amplified DNA target sequence (FIG. 1). A mediator probe is an oligonucleotide and has a sequence-specific probe portion which binds to the target sequence and is protected at the 3′ end in some preferred embodiments, and a target sequence-unspecific portion, called mediator, which does not bind to the target sequence except for a nucleotide common to mediator and probe. During primer extension by a polymerase with exonuclease activity, the mediator is cleaved from the probe, leaving the common base on the mediator (FIG. 1). The mediator is now no longer blocked by the probe segment and can now bind to the target sequence-unspecific reporter complex and be extended here. Here, the mediator binds to the receptor complex of the base strand of the target sequence-independent modular reporter complex. The mediator is then extended along the base strand by the polymerase, wherein the signaling complex is broken up in such a way that individual components and/or molecules of this complex are cleaved off, thereby initiating a signal change compared to the original state (FIGS. 5-7).

The present invention achieves the advantageous effect that once a reaction of the signal-acting or signal-initiating oligonucleotides has been optimized, in particular the hybridization reaction on the solid phase surface, it can be easily and reproducibly transferred to further target sequences. A further advantage of the invention is that the universal nature of the surface modification allows the sequences involved in the surface reactions to remain unchanged, even if these surfaces are to be used for the detection of further target sequences. In embodiments, a target sequence-specific reaction preferably takes place in the liquid phase, so that only the reagents and oligonucleotides of the liquid phase have to be adapted, newly developed and optimized for the development of a new nucleic acid detection. Furthermore, it is advantageous that preferably the surfaces and their modification (e.g. the immobilization of oligonucleotides on electrode surfaces) can be produced in large batches and thus at low cost. In summary, the invention makes it possible to save development effort (time and costs).

In embodiments of the method according to the invention, either the base strand or the at least one signal initiation oligo comprises the at least one marker.

To detect an electrochemical signal change during an amplification reaction, cleavage of the mediator probe and subsequent extension of the mediator on the target sequence-unspecific modular reporter can result in the bond of an electroactively labeled signal initiation oligo to the base strand being broken. This can then bind to an auxiliary oligo that has been immobilized on a solid phase (electrode) and thus induce a signal change (FIG. 6iii). As an alternative approach, the target sequence-independent modular reporter complex can be coupled to a solid phase via a linker on the base strand (FIG. 6i). By extending the mediator, the bond of the signal initiation strand with electroactive label or the bond of the electroactive label itself with the base strand is broken, which induces a signal change. As a further alternative, the signal initiation oligo itself can be immobilized to a surface and the entire target sequence-unspecific modular reporter complex can be coupled to the electrode via the base strand, which in this case has an electroactive modification (FIG. 6ii). By extending the mediator, the bond of the base strand to the signal initiation oligo is broken or prevented, resulting in a signal change.

The probability of hybridization to a solid phase probe can be influenced by the effective free enthalpy of the hybridization reaction and/or by different diffusion rates of molecules of different sizes (length and secondary structure of the DNA (complexes)). In general, this can be extended to other detection principles in which the signal is detected by enrichment/depletion of signaling labels on the solid phase. Among other things, fluorophores could be used for optical detection or magnetic particles for magnetoresistive detection.

In embodiments of the method according to the invention, the signal change in step g is based on the fact that the extension of the mediator sequence leads to an increased or decreased hybridization probability of the labelled oligonucleotide at the immobilized oligonucleotide and thus the label is enriched or reduced at the functionalized surface.

Non-limiting examples of the embodiments with an increased hybridization probability are illustrated in FIG. 6 iii). Non-limiting examples of the embodiments with a reduced hybridization probability are illustrated in FIG. 6 i)-ii).

In embodiments, the detection method is selected from the group consisting of electrochemical, optical, magnetoresistive, piezoelectric (e.g., vibrating quartz, surface acoustic waves), and/or surface plasmon resonance detection.

In embodiments of the method according to the invention, the at least one label is or comprises at least one electroactive label and the solid phase is an electrode.

In embodiments of the method according to the invention, a signal change is initiated in step g. by increasing or decreasing the distance between the at least one label and the solid phase in step g. Non-limiting examples of these embodiments are illustrated in FIG. 6iv) and v).

Another option is to immobilize the electroactive label on the solid phase (electrode), either via the base strand (FIG. 6iv) or the signal initiation oligo (FIG. 6v). In this case, a signal change is achieved by changing the distance between the electroactive molecule and the surface as a result of the separation of the bond between the signal initiation oligo and the base strand. Here, an extension of the mediator causes the distance to the surface either to increase (FIG. 6iv) or decrease (FIG. 6v), which leads to a signal change in both cases.

These embodiments are also not limited to electrochemical detection. Changing the distance of a fluorescent label can lead to a detectable signal change due to surface quenching effects, for example.

Another variant is detection comparable to the optical embodiment example according to FIG. 7. After hydrolysis of the labelled signal initiation oligonucleotide, the label has a detectably higher diffusion rate and can thus be detected even without enrichment by an oligonucleotide immobilized on the electrode. The effect was described, for example, in Pearce, D. M., Shenton, D. P., Holden, J., Gaydos, C. A., 2011. IEEE Trans. Biomed. Eng. 58 (3), 755-758. However, the version described therein requires the addition of an electroactively labeled hydrolysis probe after the PCR, which is then digested by an exonuclease that must also be added.

The advantage of the invention is, on the one hand, the possibility of being able to analyze suitable embodiments for signal generation more flexibly and quickly by means of the target sequence-unspecific reporter complex, thereby generating stronger and or more precisely distinguishable signals and providing a uniform mechanism for electrochemical detection than is possible with the prior art. With respect to electrochemical solid phase detection, the invention also allows the generation of universally functionalized electrode arrays. This means that it is no longer necessary to adapt the electrode modification to new target sequences and the electrode arrays can be produced in large batches and therefore more cost-effectively.

Therefore, in embodiments, the invention includes various detection methods, such as electrochemical, optical (e.g. by fluorescence, dyes, etc.), magnetoresistive, piezoelectric (e.g. Schwin quartz, surface acoustic waves) or surface plasmon resonance.

In embodiments of the method according to the invention, an optically detectable marker such as colloidal gold, colloidal silver, colloidal carbon, latex particles is coupled to the label.

In embodiments of the method according to the invention, the signal change in step g. is based on the fact that the change in the number of bound labels results in a change in the charge transfer.

In embodiments of the method according to the invention, the signal change in step g. is based on the fact that the number of bound labels results in a change in the electrode potential or the charge transfer.

In embodiments of the method according to the invention, the described change is determined as altered current or charge or potential or charge transfer resistance by means of, for example, voltammetric, amperometric, coulometric, potentiometric or impedimetric measurement methods.

Thus, in embodiments, the invention comprises electrochemical detection methods. The person skilled in the art is aware of the basic possibilities and principles of electrochemical detection.

In embodiments of the method according to the invention, the at least one label comprises at least one fluorophore. In embodiments, the at least one label of an oligonucleotide comprises at least one fluorophore wherein the oligonucleotide is bound to the solid phase. In such embodiments, quenchers can preferably be omitted.

In some embodiments of the method according to the invention, the signal change is based on the fact that the number of bound labels results in a change in fluorescence intensity.

In embodiments of the method according to the invention, the signal change is based on the fact that the change in the distance between the label and the solid phase causes a signal change. The solid phase is a (preferably metallic) surface that causes fluorescence quenching of proximal fluorophores.

In embodiments of the method according to the invention, the at least one label is a molecule for coupling further labels. Molecules that can be used for coupling are known to the person skilled in the art. Non-limiting examples are biotin-streptavidin, fluorescein-anti-fluorescein, etc.

In embodiments of the method according to the invention, the signal change is an optically perceptible change (for example by eye or camera) in the color intensity at the site of the immobilized oligonucleotides.

Thus, in embodiments, the invention comprises optical detection methods. The person skilled in the art is aware of the basic possibilities and principles of optical detection.

In embodiments of the method according to the invention, a nanoparticle (preferably a metal nanoparticle) is coupled to the label and the solid phase is a piezoelectric material which is excited to oscillate by an alternating electric field.

In some of these embodiments of the method according to the invention, the signal change in step g. is a change in the frequency and/or phase and/or attenuation of the oscillation as a function of the number of bound labels.

Thus, in embodiments, the invention comprises piezoelectric detection methods. The person skilled in the art is aware of the basic possibilities and principles of piezoelectric detection.

In embodiments of the method according to the invention, a metallic nanoparticle is/is coupled to the label and the solid phase is a metallic surface.

In embodiments of the method according to the invention, the signal change is based on a change in the refractive index at the surface due to the increase/decrease of bound labels, which can be determined by surface plasmon resonance spectroscopy.

Thus, in embodiments, the invention comprises surface plasmon resonance detection methods. The person skilled in the art is aware of basic possibilities and principles of surface plasmon resonance detection.

In embodiments of the method according to the invention, magnetic nanoparticles are coupled to the label and the signal change is based on a change in the resistance of a magnetoresistive sensor.

Thus, in embodiments, the invention comprises magnetoresistive detection methods. The person skilled in the art is familiar with the basic possibilities and principles of magnetoresistive detection.

In embodiments of the method according to the invention, the immobilized oligonucleotides may be immobilized at a plurality of defined positions of the solid phase. The immobilized oligonucleotides may differ in their sequence. In embodiments, the solid phase is or comprises an electrode array and/or solid phase array.

In embodiments of the method according to the invention, the target sequence is clearly identifiable via the characteristics of the label and/or the position of the signal change. If the detection takes place in embodiments by means of a solid phase array, the position of the detected signal can also enable identification of the target sequence.

In embodiments of the method according to the invention, the separated at least one signal initiation oligo comprises at least one electroactive label and wherein in step g. the at least one electroactive label is released, whereby it exhibits a detectably higher diffusion rate and thus initiates a signal change by increasing the electron transfer rate.

In embodiments of the method according to the invention, the at least one label is specific for the at least one target nucleic acid sequence and wherein the signal change initiated under step g. is characteristic for the at least one target nucleic acid sequence.

In embodiments of the method according to the invention, the detection of the signal change comprises an analysis of the signal change as a function of the detection temperature and/or the electric field and/or the frequency of the excitation signal.

In some preferred embodiments, a mediator probe comprises an oligonucleotide and a sequence-specific probe portion that binds to the target sequence and is protected at the 3′ end. This protection at the 3′ end may be a blocking group (protecting group), e.g., a chemical blocking or protecting group, which in some embodiments comprises a chain of three carbon atoms. Protection of the mediator probe at the 3′ end preferably prevents (unspecific) extension of the sequence strand by a polymerase during an amplification reaction. In accordance with the invention, in embodiments, the mediator probe may comprise any protecting or blocking group suitable for preventing (unspecific) extension of the mediator probe sequence strand by a polymerase during an amplification reaction. In some embodiments, the mediator probe is protected against (unspecific) polymerase extension by means other than a blocking group (protecting group) at the 3′ end.

In other embodiments, a mediator probe does not comprise a blocking group (protecting group) at the 3′ end and is not protected against (unspecific) polymerase extension.

In embodiments, a mediator probe protected at the 3′ end may comprise a “C3 spacer”. Such a C3 spacer may be a chemical blocking group, which in some embodiments comprises a chain of three carbon atoms. This “C3 spacer” thus preferably prevents (unspecific) polymerase extension of the mediator probe sequence strand. The person skilled in the art is familiar with typical and, depending on the embodiments, suitable blocking groups (protective groups). Also, based on the present disclosure of the invention, the person skilled in the art knows how to select suitable blocking groups (protecting groups) as routine adaptations of the invention described herein.

The process according to the invention for detecting DNA sequences using PCR in combination with an electrochemical readout accordingly comprises a novel system of individual DNA oligonucleotides. These form such a target sequence-independent modular reporter complex without covalent bonds. Surprisingly, this target sequence-independent modular reporter complex has all the advantages and performance characteristics of single-part target sequence-dependent DNA probes or single-part target sequence-independent detection molecules. The target sequence-independent modular reporter complex comprises at least two DNA sequences that bind specifically to each other and carry modifications that initiate signal generation during PCR. This makes the target sequence-independent reporter molecules flexible in design. A further special feature of the invention is that the reporter complexes according to the invention enable optical and electrochemical readout, wherein it is only the types of modifications that differ, and not the basic concept of the detection process. The resulting target sequence-independent modular reporter complex has a very high stability under PCR conditions in the initial state. Accordingly, the initial signal is comparable to that generated by a one-part DNA probe or a one-part target sequence-independent reporter. As a result, the entire resulting PCR detection system according to the invention has comparable performance characteristics to current PCR detection methods based on single-part detection molecules, although this complex of a target sequence-independent modular reporter has to be separated with each cycle of a PCR and has to be formed again before the signal is read out. The system according to the invention thus increases the efficiency of signal generation and is also suitable for the simultaneous detection of a plurality of DNA target genes in a PCR reaction (multiplex PCR). In addition, the complex is flexible in design and the individual components are inexpensive to develop and produce. Furthermore, the system enables the production of universal microarrays, for example for electrochemical detection. This means that the functionalization of the solid phase can take place in a very standardized manner and in large batches. The hybridization reaction on the solid phase itself is also reproducible after initial optimization, as the same oligonucleotide combinations are always involved, such that only the partial reactions in the liquid phase need to be optimized for new target sequences. This reduces the problem that otherwise parts of the (amplified) target sequence usually hybridize to a solid phase probe, whose properties (length, secondary structure, etc.) can have an influence on the hybridization efficiency and thus on the signal. The process according to the invention also does not require an additional step to generate a single strand from the (amplified) target sequence, as is often the case in the prior art. Overall, this represents a significant improvement over the prior art.

The various applications described herein are possible because the target sequence-independent modular reporter complex according to the invention is both flexible in design and the individual components are inexpensive to develop and produce. In addition, the system according to the invention enables the production of universal microarrays. Overall, this represents a significant improvement over the prior art.

In a further aspect, the invention relates to a kit for carrying out the method according to any one of the preceding claims comprising:
at least one oligonucleotide primer
at least one mediator probe
at least one signal initiation oligo
at least one basic strand
at least one buffer
PCR polymerase.

In the context of the kit according to the invention, the oligonucleotide primers, preferably at least one oligonucleotide primer pair, the at least one signal oligo, the at least one mediator and/or the at least one base strand may be configured or suitable for the specific detection of one or more different target sequences.

In embodiments, the kit according to the invention can be used for carrying out the method according to the invention.

In some embodiments, the kit can thus be used for the specific amplification and/or detection of target sequences in the method according to the invention. In preferred embodiments of the use of the kit according to the invention, the specific amplification reaction and/or detection reaction is a PCR, qPCR, real-time PCR, droplet PCR and/or digital PCR.

The embodiments described for one aspect of the invention may also be embodiments of any of the other aspects of the present invention. Accordingly, embodiments described for the method according to the invention may also be embodiments of the kit according to the invention. Furthermore, any embodiment described herein may also comprise features of any other embodiment of the invention. The various aspects of the invention are unified by, benefit from, are based on, and/or are related to the common and surprising discovery of the unexpected advantageous effects of the present method, namely optimized PCR detection of target sequences by reporter complexes which are themselves target sequence unspecific.

### DETAILED DESCRIPTION

The term “target sequence-unspecific reporter complex” describes a complex of target sequence-unspecific DNA oligonucleotides for signal generation during a PCR in the presence of the DNA target sequences.

In the context of the present invention, the term “base strand” describes a DNA oligonucleotide and part of the target sequence-unspecific reporter complex. A base strand serves as a basis for the binding of signal initiation oligos and mediators, which thus form a signaling complex and receptor complex. In the context of the present invention, the term “base strand” describes a nucleic acid oligonucleotide (e.g., a DNA oligonucleotide) and part of the target sequence-unspecific reporter complex. A base strand serves as a basis for the binding of signal initiation oligos and mediators, which thus form a signaling complex and receptor complex. Therefore, a base strand preferably comprises at least one mediator binding site and at least one signal oligo binding site. In embodiments, a base strand comprises one or more mediator binding sites and/or signal oligo binding sites. Thus, a base strand may comprise at least 1, 2, 3, 4, 5, 6, 7, 8, 9, 10, 11, 12, 13, 14, 15, 16, 17, 18, 19, 20, 25, 30, 40 or even 50 mediator binding sites. A base strand may further comprise at least 1, 2, 3, 4, 5, 6, 7, 8, 9, 10, 11, 12, 13, 14, 15, 16, 17, 18, 19, 20, 25, 30, 40 or even 50 signal oligo binding sites. In preferred embodiments, a base strand comprises between 1 and 10 mediator binding sites and between 1 and 10 signal oligo binding sites. A mediator binding site may correspond to one or more signal oligo binding sites, in other words, an (activated) mediator bound to a mediator binding site may, when extended by a (PCR) polymerase, lead to the activation or degradation, digestion, (de) cleavage or release of one or more signal oligos from one or more, preferably upstream (towards the 5′ end), signal oligo binding sites. A base strand may comprise one or more signaling complexes and one or more receptor complexes, wherein a signal oligo complex may comprise at least one signal oligo binding site and (in the non-activated state) at least one signal oligo, and a receptor complex may comprise at least one mediator binding site.

In the context of the present invention, a signal initiation oligo is a DNA oligonucleotide and part of the signal complex which, in the presence of a target sequence, initiates a signal change by itself and or parts thereof being cleaved or broken from the signal complex.

In the context of the present invention, a “signal initiation oligonucleotide”, “signal initiation oligo” or “signal oligo” for short is a nucleic acid oligonucleotide (preferably a DNA oligonucleotide) and part of the signal complex which, in the presence of a target sequence, initiates a signal change by itself and or parts thereof being cleaved or broken up from the signal complex. In the context of the present invention, the terms signal initiation oligonucleotide, signal initiation oligo and signal oligo are to be regarded as equivalent and interchangeable.

In the context of the present invention, a label may preferably be one or more electroactive labels or one or more fluorophores and/or a molecule for coupling further labels. Accordingly, in the context of the invention, in embodiments, a base strand and/or a signal initiation molecule or signal initiation oligo may carry or comprise one or more electroactive labels and/or fluorophores and/or molecules for coupling further labels.

Herein, a “mediator” refers to an oligonucleotide and part of the receptor complex that can be extended along the “base strand” by the polymerase. A “mediator probe” describes a DNA oligonucleotide that establishes the link between the target sequence and the target sequence-independent (modular) receptor by binding to a DNA target sequence during PCR in the presence of the target sequence, being cleaved by the exonuclease activity of the polymerase and releasing a mediator sequence that forms a receptor complex together with the base strand.

The term “nucleic acid” refers to nucleic acid molecules including, without limitation, DNA, SSDNA, dsDNA, RNA, mRNA, tRNA, lncRNA, ncRNA, microRNA, siRNA, rRNA, sgRNA, piRNA, rmRNA, snRNA, snoRNA, scaRNA, gRNA, or viral RNA. Nucleic acid sequences herein refer to a consecutive arrangement of nucleotides, wherein the nucleotides are represented by their nucleobases in guanine (G), adenine (A), cytosine (C) and thymine (T) in DNA and uracil (U) in RNA. A nucleic acid sequence herein may also refer to the sequence of consecutive letters or nucleobases (consisting of G, A, C and T or U) representing the actual sequence of consecutive nucleic acids in a DNA or RNA strand. This nucleic acid sequence can be identified and characterized biochemically and bioinformatically using DNA or RNA sequencing or specifically detected by complementary nucleic acid probes (e.g., in embodiments herein by mediator probes), e.g., as part of a PCR, real-time PCR or detection reaction of a digital PCR. The sequence analysis may also comprise comparing the nucleic acid sequence obtained or a detection signal specific thereto with one or more reference nucleic acid sequences and/or with the detection signals of housekeeping genes. The term nucleotide may be abbreviated as “nt”. The term base pair (two nucleobases bonded to each other via hydrogen bonds) may be abbreviated as “bp”.

In the context of the invention, a “target sequence” describes any nucleic acid sequence of interest which is to be detected by the method according to the invention. A target sequence may preferably be a DNA or RNA sequence. A target sequence may represent a part or the entire nucleic acid sequence of a target DNA. A mediator probe preferably comprises a sequence which is wholly or partially complementary to the nucleic acid sequence of the target sequence or a portion thereof. In some embodiments, this mediator probe sequence is 100%, 99%, 95%, 90% or 80% complementary to the target sequence. In some embodiments, a mediator probe can tolerate one or more mismatches to the target sequence and still bind to it. In other embodiments, the mediator probe only binds to a target sequence if it is 100% complementary to the target sequence.

The term “nucleic acid amplification reaction” refers to any process comprising an enzymatic reaction that enables the amplification of nucleic acids. A preferred embodiment of the invention relates to a polymerase chain reaction (PCR). “Polymerase chain reaction” (“PCR”) is the gold standard method for rapidly producing millions to billions of copies (full copies or partial copies) of a given DNA sample, enabling amplification of a very small DNA sample to a sufficiently large amount. PCR amplifies a specific region of a DNA strand (the DNA target sequence) depending on where the primers used bind to start the amplification reaction. Almost all PCR applications use a heat-stable DNA polymerase enzyme, such as Taq polymerase. Quantitative PCR (“qPCR”), or “real-time PCR”, is a specific form of PCR and is a standard method for detecting and quantifying a specific target sequence or quantifying gene expression levels in a sample in real time. In qPCR, fluorescently or electroactively labeled probes or nucleic acids (e.g., mediator probes) are hybridized in the PCR reaction and, in embodiments, cleaved or digested by the PCR polymerase during primer extension once they bind to a complementary sequence (e.g., a target sequence), wherein, in embodiments, the presence and amplification of target sequences is monitored in real time after or during each PCR cycle. This may also include monitoring hybridization and/or extension reactions on a solid phase at a particular time during a PCR cycle. A real-time

PCR allows the progress of an ongoing amplification reaction to be monitored as it occurs (i.e. in real time). Data are therefore collected over the entire duration of the PCR reaction and not at the endpoint as in conventional PCR. Measuring reaction kinetics in the early stages of PCR offers significant advantages over conventional PCR detection. In embodiments of real-time PCR, reactions are characterized by the time during the cycle when amplification of a target is first detected, rather than by the amount of target that has accumulated after a fixed number of cycles, as in conventional PCR. The higher the starting copy number of the nucleic acid target, the more likely a significant signal change (e.g. fluorescence, electrochemical signal) will be observed. Real-time PCR enables analysis by means of optical signals, which are used to detect a specific PCR product (the target sequence) using specific fluorochromes or fluorophores. An increase in the DNA product during a PCR therefore leads to an increase in the fluorescence intensity measured at each cycle. Using different colored labels, fluorescent probes can be used in multiplex assays to monitor a plurality of target sequences. For embodiments in which oligonucleotides are immobilized to the solid phase and amplification products interact with them, the position at which the signal change occurs can be used to distinguish a plurality of target sequences in addition to the characteristics of the label (e.g., wavelength of the fluorophore, redox potential of the electroactive molecule).

While real-time qPCR is dependent on the relative amount of target nucleic acid being determined in each amplification cycle, “digital PCR” allows the absolute amount of target nucleic acid to be determined on the basis of Poisson statistics, which is used to calculate the amount of target nucleic acid following endpoint PCR amplification. The steps prior to amplification are usually comparable or similar between digital PCR and qPCR. However, in qPCR, preferably all nucleic acid molecules are pooled and then amplified and analyzed, whereas in digital PCR, the nucleic acid molecules are preferably divided as best as possible into individual partitions (e.g. emulsion droplets, wells or gel beads), allowing PCR to proceed as a single reaction in each partition (in the case of emulsion droplets, this reaction is also often referred to as droplet PCR or digital droplet PCR) and allowing separate analysis of each partition. In digital PCR, the random division of the nucleic acid molecules into individual partitions takes place according to the Poisson distribution. When analyzing digital PCR, Poisson statistics are then applied to determine the average number of nucleic acid molecules per partition (none, one or more). Poisson statistical analysis of the number of positive and negative reactions provides a precise absolute quantification of the target sequence.

The specificity of the mediator probes also prevents interference with the measurements by primer dimers, which are undesirable potential by-products in PCR. In one embodiment, the invention relates to a method wherein the amplification is a multiplex PCR with more than one primer pair. Multiplex PCR is a variant of standard PCR in which two or more target sequences can be amplified and/or detected simultaneously in the same reaction by using at least one primer pair in the reaction.

In the context of the invention, a “signal change” preferably describes a significant, differentiable and/or characteristic change in the signal which is clearly distinguishable or different from potential base or background signals or baseline or background noise. The person skilled in the art is aware that under some test conditions in the context of electrochemical detection, non-specific base signals or baseline or background noise can occur due to non-specific processes at the electrodes, for example non-specific adsorption or ageing effects. Therefore, a signal change in the context of the invention preferably describes a significant, differentiable and/or characteristic change in the signal, and not a base or background signal or baseline or background noise. In preferred embodiments, this signal change can mean an increase in current (e.g. peak currents) and/or potential and/or impedance, in other words an increase in the signal. In some embodiments, a signal change is a decrease of the signal. The increase of a signal is preferably due to the fact that an amplification reaction increases the number of target sequence amplificates and thus the activation of associated signal complexes. The more target sequences are present and are bound by mediator probes in a PCR reaction, the more or earlier (in the case of real-time PCR) the signal increases. Preferably, the signal is proportional or approximately proportional to the amount of the corresponding target sequence for which the fluorophore signal (e.g. its color) is specific/characteristic. Since in the context of a digital PCR preferably only one target sequence is present per reaction space (e.g. partition, emulsion droplet), the signal increases with the number of target sequence amplificates per reaction space. In preferred embodiments, there is ideally a uniform distribution of max. 1 target sequence per reaction space (e.g. partition, emulsion droplet), such that when amplification begins and a similar amplification efficiency is present in all reaction spaces (containing a target sequence), the specific signal for the detection of an identical target sequence in different reaction spaces with comparable height/strength/intensity is generated during readout by digital or “droplet” PCR, which is preferably indicative of the presence and/or number of target sequence amplificates present in each reaction space. In embodiments, the intensity/strength of a respective label, preferably specific for a target sequence, as well as the maximum achievable signal strength/intensity may depend on the number of labels per signal oligo and signal complex and/or the type of label (e.g. type of fluorophores and/or quenchers).

“Fluorophore” (or fluorochrome, similar to a chromophore) is a fluorescent chemical compound that can re-emit light when excited by light.

“Quenching” refers to any process that reduces the fluorescence intensity of a given substance. Quenching is the basis for Forster resonance energy transfer (FRET) assays or static or contact quenching assays or a combination of both.

In principle, “surface quenching” is the emission-free transfer of energy between fluorophores and preferably metallic surfaces.

In the context of the invention, an “auxiliary oligo” refers to an oligonucleotide that is required as a third oligonucleotide in addition to the signal oligo and base strand in order to amplify the signal change by enrichment of the signal oligo at the solid phase.

In the context of the invention, “electroactive molecule” preferably means a redox molecule which, in the case of electrochemical detection, is reduced or oxidized at defined potentials, whereby electrons are transferred. In this context, “electroactively labeled” preferably means that the electroactive molecule is coupled to an oligonucleotide. Examples of electroactive molecules for labeling oligonucleotides include methylene blue, ferrocene, anthraquinone, etc.

### EXAMPLES

The invention is further described by the following examples. These are not intended to limit the scope of the invention, but rather represent preferred embodiments of various aspects of the invention, which are provided to illustrate the invention described herein.

### Electrochemical Detection

### Non-Labeled Solid Phase Probe

To detect an electrical signal change during an amplification reaction, cleavage of the mediator probe and subsequent extension of the mediator on the target sequence-unspecific modular reporter can result in the bond of an electroactively labeled signal initiation oligo to the base strand being broken. This can then bind to an auxiliary oligo that has been immobilized on a solid phase (electrode) and thus induce a signal change (FIG. 6 iii). As an alternative approach, the target sequence-independent modular reporter complex can be coupled to a solid phase via a linker on the base strand (FIG. 6 i). By extending the mediator, the bond of the signal initiation strand with electroactive labeling or the bond of the electroactive label itself to the base strand is broken, resulting in a signal change. As a further alternative, the signal initiation oligo itself can be immobilized on a surface and the entire target sequence-unspecific modular reporter complex can be coupled to the electrode via the base strand, which in this case has an electroactive modification (FIG. 6 ii). By extending the mediator, the binding of the base strand to the signal initiation oligo is broken, resulting in a signal change.

The probability of hybridization to a solid phase probe can be influenced by the effective free enthalpy of the hybridization reaction and/or by different diffusion rates of molecules of different sizes (length and secondary structure of the DNA (complexes)).

In general, it is conceivable that the system can be extended to other detection principles in which the signal is detected by enrichment/depletion of signaling labels on the solid phase. Among other things, fluorophores could be used for optical detection or magnetic particles for magnetoresistive detection (detecting the movement of ferromagnetic materials via the change in magnetic flux).

### Electroactive Label on Solid Phase

Another option is to immobilize the electroactive label on the solid phase (electrode), either via the base strand (FIG. 6 iv) or the signal initiation oligo (FIG. 6 v). In this case, a signal change is achieved by changing the distance between the electroactive molecule and the surface as a result of the separation of the bond between the signal initiation oligo and the base strand. Here, an extension of the mediator either causes the distance to the surface to increase (iv) or decrease (v), which leads to a signal change in both cases.

These examples are also not limited to electrochemical detection. Changing the distance of a fluorescent label can lead to a detectable signal change due to surface quenching effects, for example.

Detection without Oligo-Modified Solid Phase

A further embodiment comprises electrochemical detection comparable to optical detection, as shown, for example, in FIG. 7. After hydrolysis of the labeled signal initiation oligonucleotide, the label exhibits a detectably higher diffusion rate and can thus be detected even without enrichment by an oligonucleotide immobilized on the electrode (the effect was described, for example, in Pearce, D. M., Shenton, D. P., Holden, J., Gaydos, C. A., 2011. IEEE Trans. Biomed. Eng. 58 (3), 755-758. However, in the version described there).

### Results

To demonstrate a detection reaction according to the reporter complex setup as shown in FIG. 6 i), a mediator extension reaction was performed in the presence of the electrode surface functionalized with the base strand. The reaction mix comprised a PCR mix (incl. polymerase, co-factors, etc.) as well as 300 nM of the electroactively labeled molecule (signal oligo) and also 200 nM of either the mediator or the intact mediator probe. The sequences of the oligonucleotides are listed in Table 1. The reaction mix was initially incubated for 5 min at 95° C. to activate the polymerase.

The reaction mix was then transferred to reaction chambers of a functionalized electrode array. The closed reaction chambers were first incubated in a water bath at 40° C. and then heated up to 62° C. During the heating phase and the subsequent cooling process, the electrodes were analyzed electrochemically using square-wave voltammetry. When the electroactive molecule is enriched on the electrode surface through hybridization, the current signal exhibits a characteristic peak (Δi_{Peak}).

TABLE 1
Sequences for the mediator extension reaction according to FIG. 6
i). Sequence segments belonging to the receptor complex are shown in italics.
Sequence segments belonging to the signal complex are underlined.

| OLIGONUCLEO- | | |
|---|---|---|
| TIDE | SEQUENCE 5′→3′ | MODIFICATION |
| MEDIATOR PROBE | TGCTC CAGTT CGGTC AGTTT GCCCG | 3′: C3 spacer |
| | CATTG CATTA GCATT AGGA | |
| MEDIATOR | ATGCT CCAGT TCGGT CAGT | |
| BASE STRAND | ttttt ATCCG ACGTT GGCAT GTGAG TATGT | 5′: Thiol |
| | TCACT GACCG AACTG GAGCA | |
| SIGNAL OLIGO | CTCAC ATGCC AACGT CGGAT | 3′: Methylene blue |

The result (FIGS. 2 and 3) initially shows electrochemical signals that are easily detectable at temperatures below 45° C., but which do not differ between the different reaction mixes. This indicates hybridization of the signal oligo to the solid phase probe, but no significant displacement reaction of an extended mediator. With increasing temperature and thus increasing activity of the polymerase, the signal for reactions containing mediators starts to decrease and to differ from reactions with mediator probe. This indicates that the mediator on the solid phase oligo is extended by polymerase activity, displacing the electroactively labeled oligonucleotide from it. Even after or during the cooling process, the signals from reaction preparations with active mediator remain distinguishable from the reaction preparations with mediator probes (FIG. 3).

For a real PCR (real-time PCR), the active mediator must be generated by cleavage of the mediator probe. In this case-in addition to polymerase, PCR buffer and 200 nM mediator probe-target sequence-specific primers (>200 nM) are added to the PCR mix as well as a target DNA for positive controls (PTC) that contains the target sequence to be amplified, while the “no template control” (NTC) to be carried out as well does not contain any target DNA. The base strand is immobilized on the surface of the working electrode. The signal oligo can be initially present in a pre-hybridized state with the base strand or be part of the PCR mix. In real-time detection, the PCR takes place in a chamber containing the functionalized electrode. In the case of endpoint PCR detection, the amplification reaction can also take place in an upstream chamber. The PCR is carried out according to the recommended temperature protocol for the polymerase used, i.e. after activation of the hotstart polymerase by cyclic temperature changes between 95° C. and approx. 60° C. The signal is detected during the approx. 60° C. temperature step (typical temperature for annealing primers and probes to target sequences). By cleaving the mediator probe in the course of the PCR, increasingly activated mediators are generated which hybridize to the base strand and lead to a detectable displacement of the electroactively labelled signal oligo by extension or prevent its rehybridization to the base strand in the next temperature step. This causes a decrease in signal; in the case of endpoint detection, possibly in comparison to NTC reactions that have uncleaved mediator probes. A prerequisite for real-time detection is that the melting temperature (Tm) of the mediator and signal oligo at their respective binding sites is above the evaluation temperature. In the case of endpoint detection, there is greater freedom with regard to the melting temperature, as the mediator extension and signal oligo hybridization can take place below the primer annealing temperature. In this case, the results to date indicate that sufficient polymerase activity for the mediator extension reaction and signal oligo displacement reaction is given up to a temperature of 45° C., i.e. an extended temperature window between 45° C. and approx. 60° C. (even higher if the Tm of mediator and signal oligo is configured accordingly) is possible.

For a detection reaction according to the reporter complex setup as shown in FIG. 6 ii), a mediator extension reaction was carried out under PCR conditions in a standard PCR cycler. The electroactively labeled molecule (base strand) and the mediator or the intact mediator probe were added to the PCR reagents as a control. After 10 cycles, the reaction result was transferred to an electrode array functionalized with the signal oligo. The hybridization reaction taking place there at room temperature was analyzed electrochemically using square-wave voltammetry. The sequences used are listed in Table 2.

The result (FIG. 4) shows an increasing electrochemical signal for reactions that contained an intact mediator probe (dark gray (upper) curve). Surprisingly, no electrochemical signals are detectable for reactions that contained mediators (light gray (lower) curve). This indicates an effective extension reaction of the mediator on the electroactively labelled strand, which subsequently leads to an effective prevention of the hybridization reaction on the functionalized electrode surface. An increased temperature or polymerase activity is no longer absolutely necessary for the detection step, wherein the hybridization speed would increase from a higher hybridization temperature and the signal plateau would be reached more quickly.

TABLE 2
Sequences for the mediator extension reaction according to FIG. 6
i). Sequence segments belonging to the receptor complex are shown in italics.
Sequence segments belonging to the signal complex are underlined.

| OLIGONUCLEO- | | |
|---|---|---|
| TIDE | SEQUENCE 5′→3′ | MODIFICATION |
| MEDIATOR PROBE | TGCTC CAGTT CGGTC AGTTT GCCCG | 3′: C3 spacer |
| | CATTG CATTA GCATT AGGA | |
| MEDIATOR | ATGCT CCAGT TCGGT CAGT | |
| BASE STRAND | CCAAG ACGCG CCGGT CTGTT CACTG | 5′: Methylene blue |
| | ACCGA ACTGG AGCA | 3′: C3 spacer |
| SIGNAL OLIGO | GTCAG TGAAC AGACC GGCGC GTCTT | 3′: Thiol |
| | GGttt tt | |

For a real PCR, the addition of target sequence-specific primers to the aforementioned reaction components (polymerase, buffer, electroactively labeled base strand) is also necessary in this case, as well as target DNA for the PTC. PTC and NTC reactions are prepared and PCR cycles (between 95° C. and approx. 60° C.) are carried out. For real-time detection, the electrode functionalized with signal oligo is in the reaction chamber. This is not absolutely necessary for endpoint detection. In the PTC, mediator probes are cleaved in the course of the PCR during the primer extension reaction. The activated mediator hybridizes to the base strand and is extended there, such that the base strand is separated from the immobilized signal oligo and/or its rehybridization to the signal oligo is prevented. Signal detection in real-time PCR takes place at the end of each primer/mediator extension step at approx. 60° C. A decrease in signal is observed in the PTC. As previously mentioned, the melting temperatures of the signal complex must be close to or above the evaluation temperature. In the case of endpoint detection, the signal of the PTC is lower than that of the NTC reactions. The evaluation temperature can be selected from room temperature to the melting temperature of the signal complex, wherein a higher temperature is preferable due to the faster hybridization speed and the avoidance of unspecific hybridization processes.

### FIGURES

FIG. 1: The figure shows the mediator-probe cleavage during a PCR with activation of the mediator in one embodiment of the invention. The “activated” mediator here corresponds to a mediator sequence which is released after digestion of the mediator probe.

FIG. 2: Electrochemical signal as a function of time for carrying out a mediator extension reaction according to the example in FIG. 6 i) in the presence of the mediator (active) or the mediator probe. The peak currents (Δi_{Peak}) were normalized at time t=5 min. The temperature shown refers to the temperature of the water bath.

FIG. 3: Quotient of normalized electrochemical signal of the reactions with mediator to reactions with mediator probe (compare FIG. 2) as a function of the temperature of the water bath. The diagram distinguishes between signals recorded during the heating phase (squares) and during the cooling phase (crosses).

FIG. 4: Comparison of the electrochemical signal of a hybridization reaction after thermocycling in the presence of electroactively labelled oligonucleotides and mediators (light grey) or intact mediator probes (dark grey) according to FIG. 6 ii).

FIG. 5: The figure shows an example of electrochemical signal generation after mediator activation using a target sequence-unspecific modular reporter complex.

FIG. 6: The figure shows an example of electrochemical signal generation after mediator activation using a target sequence-independent modular reporter complex. In one embodiment, the target sequence-independent modular reporter complex can be coupled to a solid phase via a linker on the base strand (FIG. 6i). By extending the mediator, the bond of the signal initiation strand with electroactive label or the bond of the electroactive label itself to the base strand is broken, resulting in a signal change. In a further embodiment, the signal initiation oligo itself can be immobilized to a surface and the entire target sequence-unspecific modular reporter complex can be coupled to the electrode via the base strand, which in this case has an electroactive modification (FIG. 6ii). In a further embodiment, a signal initiation oligo can bind to an auxiliary oligo that has been immobilized on a solid phase (electrode) such that a signal change is induced (FIG. 6iii). Two further embodiments are the immobilization of the electroactive label on the solid phase (electrode), either via the base strand (FIG. 6iv) or the signal initiation oligo (FIG. 6v). In this case, a signal change is achieved by changing the distance between the electroactive molecule and the surface as a result of the separation of the bond between the signal initiation oligo and the base strand. Here, an extension of the mediator either causes the distance to the surface to increase (FIG. 6iv) or decrease (FIG. 6v), which leads to a signal change in both cases.

FIG. 7: The figure shows a further embodiment, wherein the detection does not require an immobilized oligonucleotide. After hydrolysis of the labelled signal initiation oligonucleotide, the label exhibits a detectably higher diffusion rate and can thus be detected even without enrichment by an oligonucleotide immobilized on the electrode.

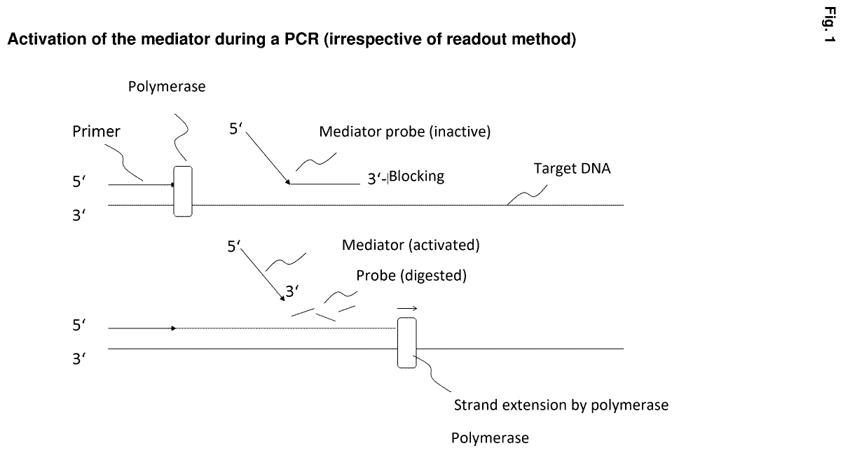

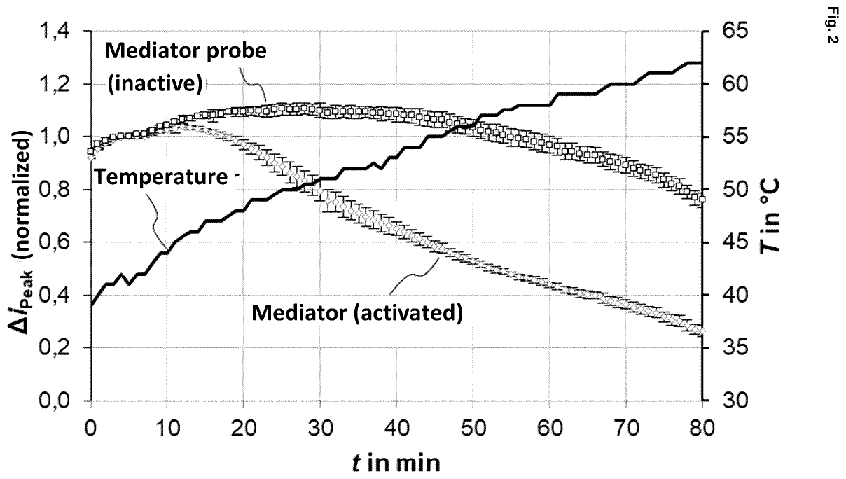

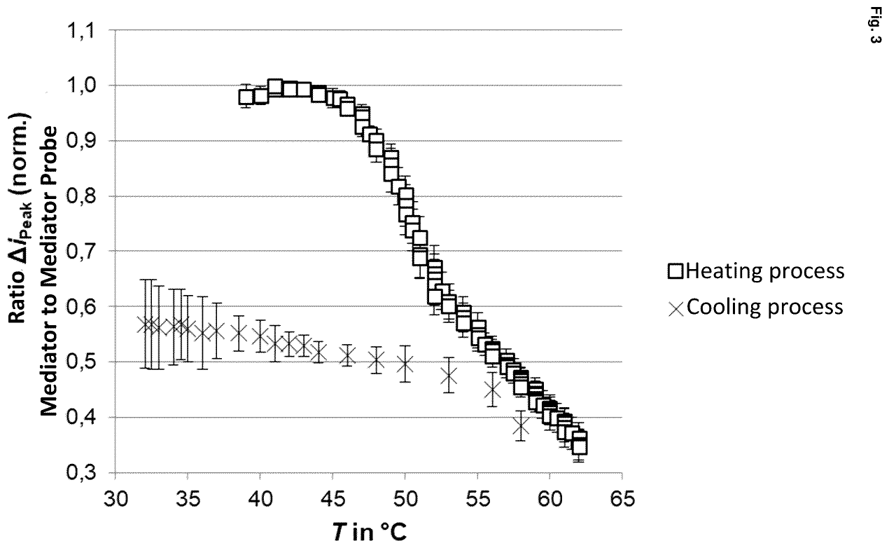

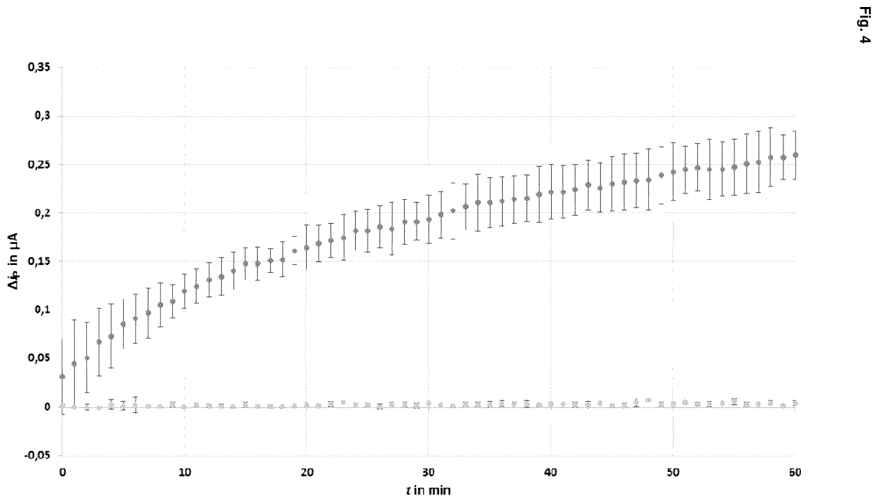

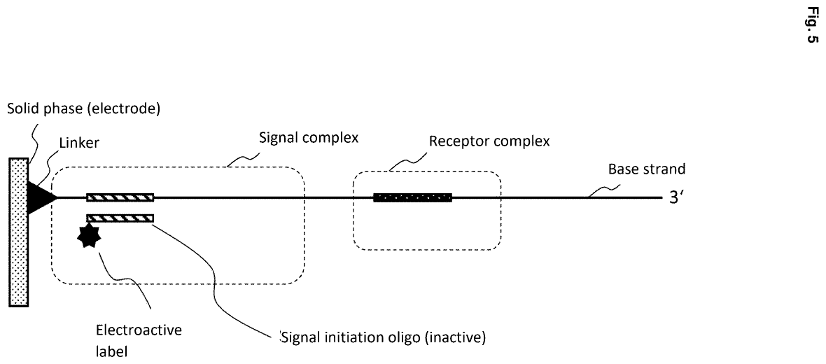

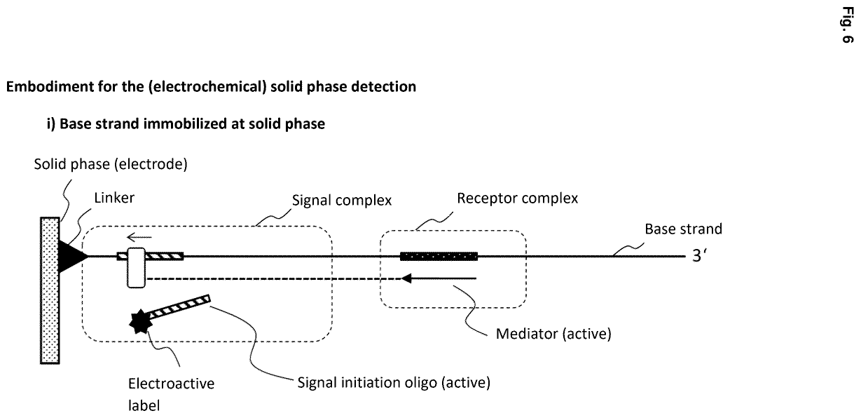

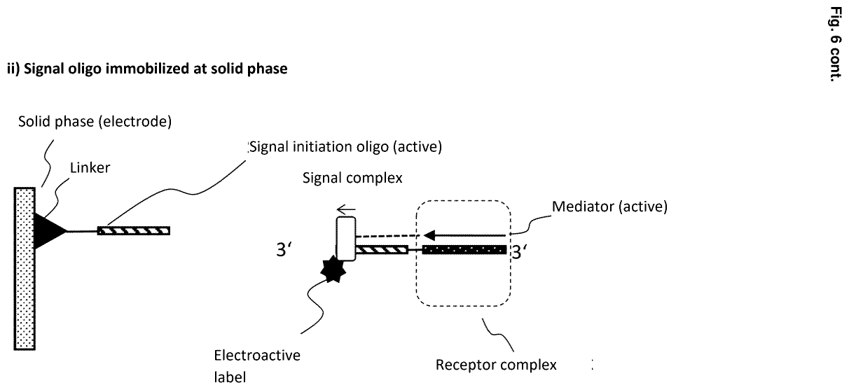

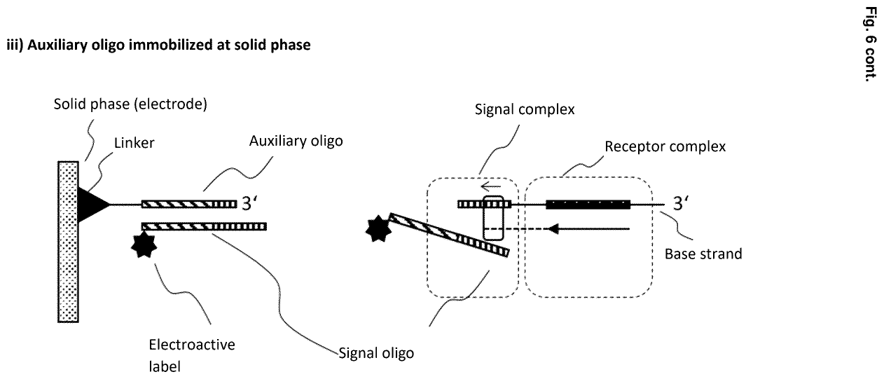

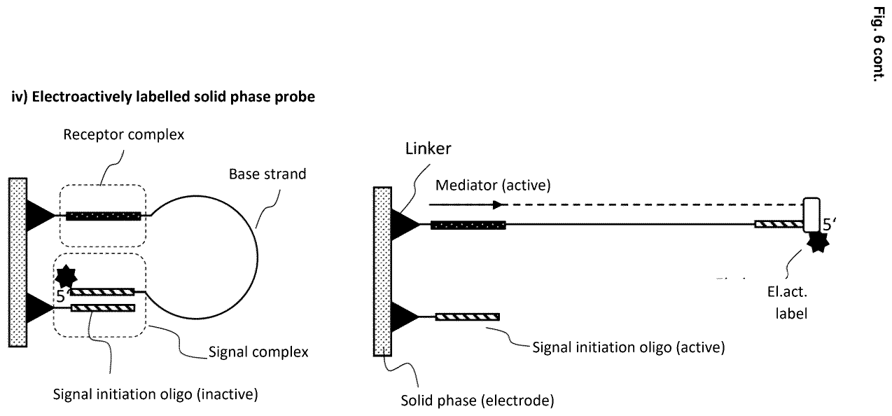

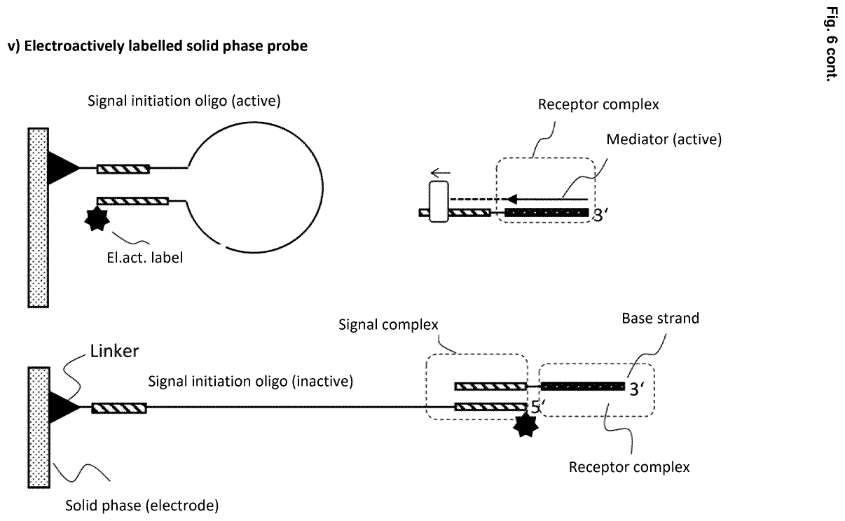

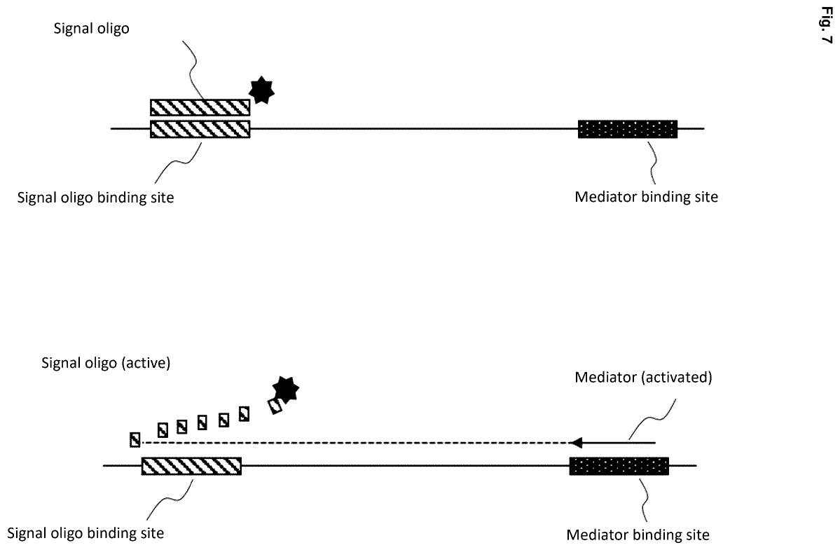

### REFERENCES

Becherer, Lisa; Bakheit, Mohammed; Frischmann, Sieghard; Stinco, Silvina; Borst, Nadine; Zengerle, Roland; Stetten, Felix von (2018): Simplified Real-Time Multiplex Detection of Loop-Mediated Isothermal Amplification Using Novel Mediator Displacement Probes with Universal Reporters. In: Anal. Chem. 90 (7), pp. 4741-4748. DOI: 10.1021/acs.analchem.7b05371.
Faltin, Bernd; Wadle, Simon; Roth, Günter; Zengerle, Roland; Stetten, Felix von (2012): Mediator probe PCR: a novel approach for detection of real-time PCR based on label-free primary probes and standardized secondary universal fluorogenic reporters. In: Clinical Chemistry 58 (11), pp. 1546-1556. DOI: 10.1373/clinchem.2012.186734.
Faltin, Bernd; Zengerle, Roland; Stetten, Felix von (2013): Current Methods for FluorescenceBased Universal Sequence-Dependent Detection of Nucleic Acids in Homogenous Assays and Clinical Applications. In: Clinical Chemistry 59 (11), pp. 1567-1582. DOI: 10.1373/clinchem.2013.205211.
Heid, C. A.; Stevens, J.; Livak, K. J.; Williams, P. M. (1996): Real time quantitative PCR. In: Genome Research 6 (10), pp. 986-994. DOI: 10.1101/gr.6.10.986.
Holland, P. M.; Abramson, R. D.; Watson, R.; Gelfand, D. H. (1991): Detection of specific polymerase chain reaction product by utilizing the 5′- - - 3′ exonuclease activity of Thermus aquaticus DNA polymerase. In: Proceedings of the National Academy of Sciences of the United States of America 88 (16), pp. 7276-7280. DOI: 10.1073/pnas.88.16.7276.
Lehnert, Michael; Kipf, Elena; Schlenker, Franziska; Borst, Nadine; Zengerle, Roland; Stetten, Felix von (2018): Fluorescence signal-to-noise optimization for real-time PCR using universal reporter oligonucleotides. In: Anal. Methods 10 (28), pp. 3444-3454. DOI: 10.1039/C8AY00812D.
Li, Yongsheng; Zhou, Xiaoyan; Ye, Duyun (2008): Molecular beacons: an optimal multifunctional biological probe. In: Biochemical and biophysical research communications 373 (4), pp. 457-461. DOI: 10.1016/j.bbrc.2008.05.038.
Liu, Shufeng; Fang, Li; Wang, Yanqun; Wang, Li (2017): Universal Dynamic DNA Assembly-Programmed Surface Hybridization Effect for Single-Step, Reusable, and Amplified Electrochemical Nucleic Acid Biosensing. In: Anal. Chem. 89 (5), pp. 3108-3115. DOI: 10.1021/acs.analchem.6b04871.
Lyamichev, V.; Brow, M. A.; Dahlberg, J. E. (1993): Structure-specific endonucleolytic cleavage of nucleic acids by eubacterial DNA polymerases. In: Science (New York, N.Y.) 260 (5109), pp. 778-783. DOI: 10.1126/science.7683443.
Rodríguez, Miguel A.; García, Teresa; González, Isabel; Hernández, Pablo E.; Martín, Rosario (2005): TaqMan real-time PCR for the detection and quantitation of pork in meat mixtures. In: Meat science 70 (1), pp. 113-120. DOI: 10.1016/j.meatsci.2004.12.005.
Tan, Weihong; Wang, Kemim; Drake, Timothy J. (2004): Molecular beacons. In: Current opinion in chemical biology 8 (5), pp. 547-553. DOI: 10.1016/j.cbpa.2004.08.010.
Tyagi, S.; Kramer, F. R. (1996): Molecular beacons: probes that fluoresce upon hybridization. In: Nat Biotechnol 14 (3), pp. 303-308. DOI: 10.1038/nbt0396-303.
Wadle, Simon; Lehnert, Michael; Schuler, Friedrich; Köppel, René; Serr, Annerose; Zengerle, Roland; Stetten, Felix von (2016): Simplified development of multiplex real-time PCR through master mix augmented by universal fluorogenic reporters. In: BioTechniques 61 (3), pp. 123-128. DOI: 10.2144/000114443.
Whale, Alexandra S.; Huggett, Jim F.; Tzonev, Svilen (2016): Fundamentals of multiplexing with digital PCR. In: Biomolecular Detection and Quantification 10, pp. 15-23. DOI: 10.1016/j.bdq.2016.05.002.

1. A method for detecting at least one target nucleic acid sequence, comprising:
a) providing at least one target-sequence-unspecific modular reporter complex comprising at least one label and
at least two oligonucleotides comprising:
i. a base strand, comprising:
at least one mediator binding site and
at least one signal initiation oligo binding site, and
ii. at least one signal initiation oligo,
b) providing at least one mediator probe, wherein the mediator probe comprises an oligonucleotide having at least one probe sequence and at least one mediator sequence, wherein
the at least one probe sequence exhibits an affinity for the at least one target nucleic acid sequence, and the at least one mediator sequence has an affinity for the at least one mediator binding site on the base strand of the at least one target-sequence-unspecific modular reporter complex,
c) conducting a PCR amplification of the at least one target nucleic acid sequence,
d) binding the probe sequence of the at least one mediator probe to the at least one target nucleic acid sequence,
e) cleaving the probe sequence of the at least one mediator probe bound to the at least one target nucleic acid sequence via a PCR polymerase, wherein the mediator sequence is released resulting in a at least one released mediator sequence,
f) binding the at least one released mediator sequence to the at least one mediator binding site of the at least one target-sequence-unspecific modular reporter complex,
g) extension of the sequence of at least one mediator sequence bound to the at least one mediator binding site via a PCR polymerase, wherein the hybridization bond of the at least one signal initiation oligo binding site and the at least one signal initiation oligo is either i) broken and/or prevented or ii) initiated and/or produced, thereby initiating a signal change and resulting in at least one separated signal initiation oligo,
h) detection of at least one signal change as evidence of the at least one target nucleic acid sequence.

2. The method according to claim 1 further comprising a solid phase, wherein in g) the signal change is initiated by:
the at least one separated signal initiation oligo comprising the at least one label resulting in a labelled oligonucleotide and binding to at least one auxiliary oligo which is immobilized on the solid phase, or
one of the two oligonucleotides or both of the oligonucleotides of the at least one target-sequence-unspecific modular reporter complex comprising:
i. the base strand, and/or
ii. the at least one signal initiation oligo,
being immobilized to the solid phase respectively via a linker, wherein the immobilization creates a functionalized surface comprising immobilized oligonucleotides.

3. (canceled)

4. The method according to claim 2, wherein either the base strand or the at least one signal initiation oligo comprises the at least one label.

5. The method according to claim 4, wherein the signal change in g) is based on the extension of the mediator sequence leads to an increased or decreased probability of the labelled oligonucleotide hybridizing to the immobilized oligonucleotide and thus the label being enriched or reduced at the functionalized surface.

6. The method according to claim 2, wherein the at least one label comprises at least one electroactive label and the solid phase is an electrode.

7. The method according to claim 6, wherein in g):
the signal change is initiated by increasing or decreasing the distance between the at least one label and the solid phase in g), or
the signal change is based on a number of bound labels resulting in a change of the electrode potential or the charge transfer.

8. (canceled)

9. The method according to claim 7, wherein the change is determined as altered current or charge or potential or charge transfer resistance via, optionally, voltammetric, amperometric, coulometric, potentiometric or impedimetric measuring methods.

10. The method according to claim 2, wherein
the at least one label comprises at least one fluorophore.

11. The method according to claim 10, wherein the signal change is based on:
a number of bound labels resulting in a change in fluorescence intensity, or
the change in a distance between the label and the solid phase causing the signal change, wherein the solid phase is a, optionally metallic, surface which causes fluorescence quenching of proximal fluorophores.

12. (canceled)

13. The method according to claim 2, wherein the at least one label is a molecule for coupling further labels.

14. The method according to claim 13, wherein an optically detectable marker, optionally colloidal gold, colloidal silver, colloidal carbon, latex particles, is coupled to the label and wherein the signal change is optionally optically perceptible change in color intensity at the location of the immobilized oligonucleotides.

15. (canceled)

16. The method according to claim 13, wherein a nanoparticle, optionally a metal nanoparticle, is coupled to the label and the solid phase is a piezoelectric material which is excited to oscillate by an alternating electric field and wherein the signal change in g) is optionally a change in frequency and/or phase and/or attenuation of the oscillation depending on a number of bound labels.

17. (canceled)

18. The method according to claim 13, wherein a metallic nanoparticle is coupled to the label and the solid phase is a metallic surface, wherein the signal change is optionally based on a change in the refractive index at the surface due to the increase/decrease of bound labels, which can be determined via surface plasmon resonance spectroscopy.

19. (canceled)

20. The method according to claim 13, wherein magnetic nanoparticles are coupled to the label and the signal change is based on a change in the resistance of a magnetoresistive sensor.

21. The method according to claim 2, wherein the immobilized oligonucleotides can be immobilized at a plurality of defined positions of the solid phase wherein the immobilized oligonucleotides may differ in their sequence.

22. The method according to claim 1, wherein the target sequence is uniquely identifiable by characteristics of the label and/or a position of the signal change.

23. The method according to claim 1, wherein at least one separated signal initiation oligo comprises at least one electroactive label and wherein in g) the at least one electroactive label is released, whereby it has a detectably higher diffusion rate and thus initiates a signal change, optionally by an increase in the electron transfer rate.

24. The method according to claim 1, wherein the at least one label is specific for the at least one target nucleic acid sequence and wherein the signal change initiated under g) is characteristic for the at least one target nucleic acid sequence.

25. The method according to claim 1, wherein the detection of the signal change comprises an analysis of the signal change as a function of a detection temperature and/or a electric field and/or a frequency of an excitation signal.

26. The method according to claim 1, wherein in a) the signal initiation oligo binding site of the base strand and the at least one signal initiation oligo are hybridized to each other, but not covalently bonded, and together form a signal complex.

27. A kit for carrying out the method according to claim 1, comprising:
at least one oligonucleotide primer,
the at least one mediator probe,
the at least one signal initiation oligo,
the at least one base strand,
at least one buffer, and
PCR polymerase.
